(19) United States
(12) Patent Application Publication
(10) Pub. No.: US 2026/0153506 A1
(43) Pub. Date: Jun. 4, 2026

(54) PARTICLE FOR IMMUNOTURBIDIMETRY, REAGENT, TEST KIT, AND METHOD OF DETECTING TARGET SUBSTANCE

(71) Applicant: CANON KABUSHIKI KAISHA, Tokyo (JP)

(72) Inventors: RYO NATORI, Tokyo (JP); FUMIO YAMAUCHI, Kanagawa (JP); KENGO KANAZAKI, Kanagawa (JP); GOTA KASAI, Tokyo (JP)

(21) Appl. No.: 19/396,512

(22) Filed: Nov. 21, 2025

(30) Foreign Application Priority Data
Nov. 29, 2024 (JP) 2024-208571

(51) Int. Cl. G01N 33/553; G01N 33/68

(52) U.S. Cl. CPC G01N 33/553; G01N 33/68

(57) ABSTRACT

Provided are a particle for immunoturbidimetry, a reagent, a test kit, and a method of detecting a target substance that can each detect a trace component in a low-concentration region and suppress a change in water dispersion stability over time. The particle for immunoturbidimetry is a particle for immunoturbidimetry including a first resin and titanium oxide, wherein the particle for immunoturbidimetry includes a first layer containing the first resin and a second layer containing the titanium oxide, wherein the second layer is arranged inside the first layer, and wherein the first resin has at least one of a structure represented by the formula (1-1) and a structure represented by the formula (1-2).

### BACKGROUND

### Field of the Technology

The present disclosure relates to a particle for immunoturbidimetry, a reagent, a test kit, and a method of detecting a target substance.

### Description of the Related Art

In recent years, immunoturbidimetry has been drawing attention as a simple and rapid immunological testing method. As the immunoturbidimetry, there has been known a method including mixing a dispersion of a particle having an antibody or an antigen as a ligand on its surface and a specimen that may contain a target substance (antigen or antibody). At this time, when the specimen contains the target substance (antibody or antigen), the particle causes an agglutination reaction, and hence the presence or absence of a disease can be identified by optically detecting the agglutination reaction as a variation in, for example, scattered light intensity, transmitted light intensity, or absorbance.

A polystyrene-based latex particle containing polystyrene as a main component has hitherto been used as a particle for immunoturbidimetry because the polystyrene-based latex particle is easy for sensitization (immobilization) of an antigen or an antibody and is relatively inexpensive, and its polymerization reaction is easy to control.

For example, there has been developed a particle using titanium oxide, which is well known as a high-refractive-index material. In Japanese Patent Laid-Open No. 2017-122684, there is a proposal of a particle for immunoturbidimetry in which a titanium oxide fine particle is coated with a polyethylene glycol analog.

### SUMMARY

In the immunoturbidimetry using the polystyrene-based latex particle, a problem has arisen in that a trace component in a low-concentration region cannot be detected in some cases. Accordingly, the development of a particle having sensitivity higher than that of the polystyrene-based latex particle has been demanded.

In order to solve the above-mentioned problem, it is required to increase a change in absorbance caused by particle aggregation along with the formation of an immune complex. The change in absorbance is known to be related to the refractive index of a component for forming a particle, there has been developed a particle using a high-refractive-index material.

In a case where titanium oxide is used as a particle for immunoturbidimetry, in addition to the suppression of non-specific adsorption, it is required to stably disperse the particle in water over a long time period. Accordingly, dispersion treatment with a resin, that is, a surfactant or a coating is performed. However, depending on the kind of the resin, the resin may be desorbed, and the water dispersion stability of the particle may decrease over time.

The present disclosure has been made in view of the background art and the problem. Specifically, the present disclosure is directed to provide a particle for immunoturbidimetry, a reagent, a test kit, and a method of detecting a target substance that can each detect a trace component in a low-concentration region and suppress a change in water dispersion stability over time in immunoturbidimetry.

That is, the present disclosure relates to the following contents.

A particle for immunoturbidimetry including a first resin and titanium oxide, wherein the particle for immunoturbidimetry includes a first layer containing the first resin and a second layer containing the titanium oxide, wherein the second layer is arranged inside the first layer, and wherein the first resin has at least one of a structure represented by the following formula (1-1) and a structure represented by the following formula (1-2):

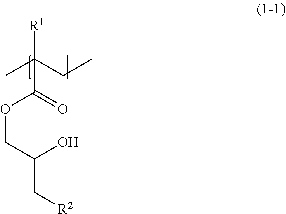

in the formula (1-1), R¹ represents a hydrogen atom or a methyl group, and R² represents a structure having a hydroxy group or a structure having a carboxy group; and

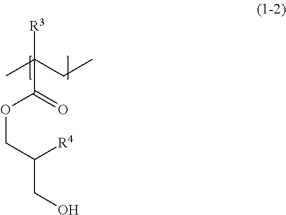

in the formula (1-2), R³ represents a hydrogen atom or a methyl group, and R⁴ represents a structure having a hydroxy group or a structure having a carboxy group.

In addition, a reagent according to the present disclosure is a reagent including the above-mentioned particle for immunoturbidimetry and an aqueous solution, wherein the particle for immunoturbidimetry is dispersed in the aqueous solution. In addition, a test kit according to the present disclosure is a test kit including the above-mentioned reagent and a container configured to accommodate the above-mentioned reagent.

In addition, a method of detecting a target substance according to the present disclosure is a method of detecting a target substance in a specimen by in vitro diagnosis, the method including mixing the above-mentioned reagent and a specimen that may contain the target substance. In addition, a method of detecting a target substance according to the present disclosure is a method of detecting a target substance in a specimen by in vitro diagnosis, the method including: mixing the above-mentioned reagent and a specimen that may contain the target substance to provide a mixed solution; irradiating the mixed solution with light; and detecting at least one of transmitted light and scattered light from the light with which the mixed solution has been irradiate d.

Features of the present disclosure will become apparent from the following description of embodiments. The following description of embodiments is described by way of example.

### DESCRIPTION OF THE EMBODIMENTS

Embodiments of the present disclosure are described in detail below. However, the technical scope of the present disclosure is not limited to the embodiments.

A particle for immunoturbidimetry according to the present disclosure is a particle for immunoturbidimetry including a first resin and titanium oxide, wherein the particle for immunoturbidimetry includes a first layer containing the first resin and a second layer containing the titanium oxide, wherein the second layer is arranged inside the first layer, and wherein the first resin has at least one of a structure represented by the following formula (1-1) and a structure represented by the following formula (1-2):

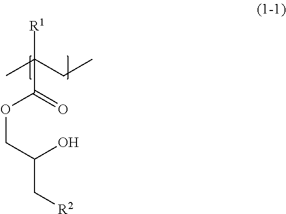

in the formula (1-1), R¹ represents a hydrogen atom or a methyl group, and R² represents a structure having a hydroxy group or a structure having a carboxy group; and

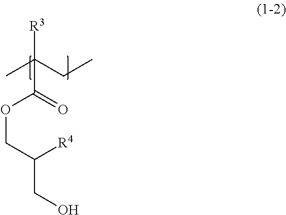

in the formula (1-2), R³ represents a hydrogen atom or a methyl group, and R⁴ represents a structure having a hydroxy group or a structure having a carboxy group.

The inventors have found that, in a particle for immunoturbidimetry including titanium oxide, a highly hydrophilic resin layer can be formed uniformly and firmly on the surface layer of the titanium oxide-containing particle by arranging a specific structure on the surface layer of the particle. This has enabled the detection of a trace component in a low-concentration region, the suppression of a change in water dispersion stability over time, and the suppression of non-specific adsorption.

The particle for immunoturbidimetry according to the present disclosure includes the first layer containing the first resin having at least one of the structure represented by the formula (1-1) and the structure represented by the formula (1-2) on the outside of the second layer containing titanium oxide. The structure of the resin is not particularly limited as long as the resin has the structure represented by the formula (1-1) or (1-2), but this structure is a vinyl-based polymer having a carboxy group or a hydroxy group in its side chain. It is conceived that, when the resin has a carboxy group or a hydroxy group in the side chain, a hydrogen bond can be formed with titanium oxide to allow for satisfactory interaction. Further, it is conceived that the resin is a vinyl-based polymer, and hence uniform coating can be performed without exposure of the titanium oxide. That is, by virtue of the presence of the structure represented by the formula (1-1) or (1-2), the coating resin (first resin) satisfactorily interacts with titanium oxide and uniform coating can be performed without exposure of the titanium oxide. Accordingly, it is possible to suppress the desorption of the coating resin, suppress a decrease in water dispersion stability of the particle for immunoturbidimetry, and suppress non-specific adsorption. Specific examples of the structure represented by the formula (1-1) or (1-2) are shown in the following formulae (1-A-1) to (1-A-12), but the present disclosure is not limited thereto.

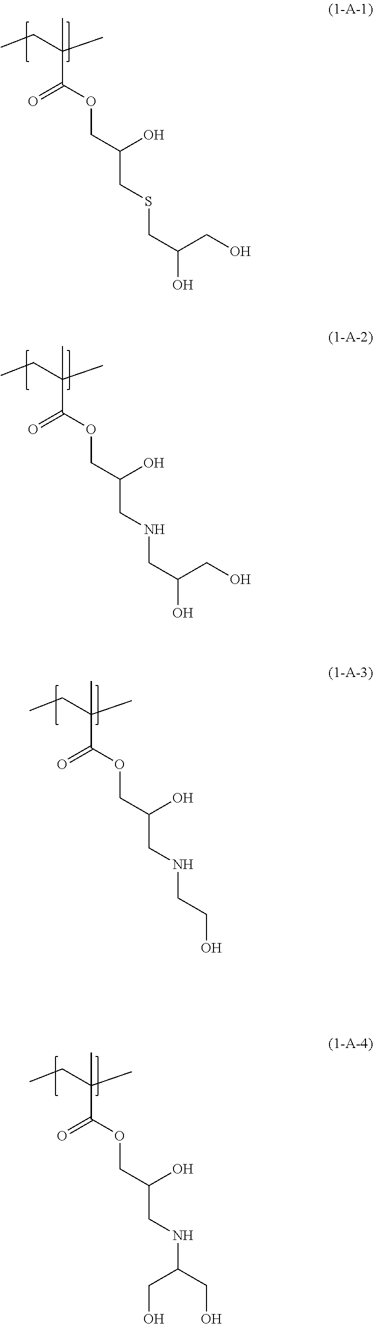

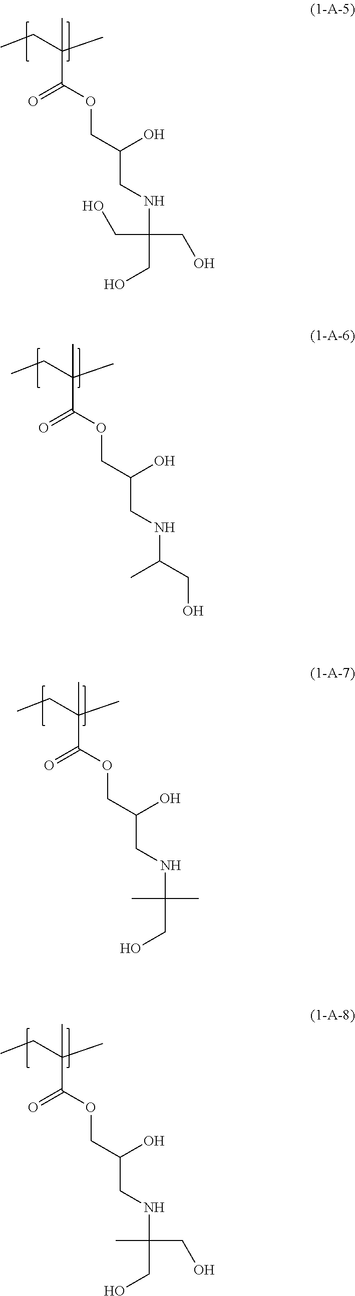

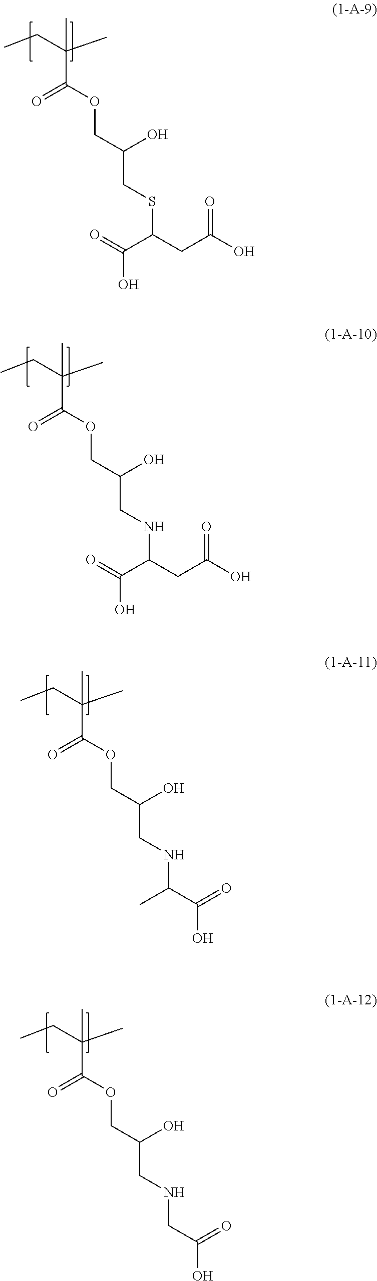

Further, the first resin preferably has at least one of a structure represented by the following formula (2) and a structure represented by the following formula (3).

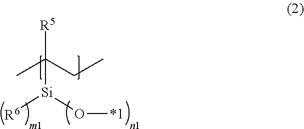

In the formula (2), R⁵ represents a hydrogen atom or a methyl group, n1 represents an integer of 1 or more and 3 or less, m1 represents an integer of 0 or more and 2 or less, n1+m1 is 3, *1s each independently represent a bond to a titanium atom or a silicon atom, or represent a hydrogen atom, a methyl group, or an ethyl group, and R⁶s each independently represent a methyl group or an ethyl group. That is, the structure represented by the formula (2) may be bonded to a titanium atom of titanium oxide via an oxygen atom, or may be bonded to a silicon atom of another structure represented by the formula (2) or the structure represented by the formula (3) via an oxygen atom.

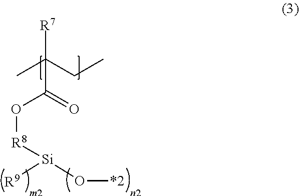

In the formula (3), R⁷ represents a hydrogen atom or a methyl group, R⁸ represents a single bond, a phenylene group, or an alkylene group having 3 or less carbon atoms, n2 represents an integer of 1 or more and 3 or less, m2 represents an integer of 0 or more and 2 or less, n2+m2 is 3, *2s each independently represent a bond to a titanium atom or a silicon atom, or represent a hydrogen atom, a methyl group, or an ethyl group, and R⁹s each independently represent a methyl group or an ethyl group. That is, the structure represented by the formula (3) may be bonded to a titanium atom of titanium oxide via an oxygen atom, or may be bonded to a silicon atom of another structure represented by the formula (3) or the structure represented by the formula (2) via an oxygen atom.

The resin further having the structure represented by the formula (2) or the structure represented by the formula (3) in addition to the structure represented by the formula (1-1) or the structure represented by the formula (1-2) is a vinyl-based polymer and has an alkoxysilane. The resin is preferred because the exposure of the titanium oxide is further suppressed by the interaction with the titanium oxide and uniform coating is achieved.

The particle for immunoturbidimetry according to the present disclosure is not particularly limited as long as the particle has the structure represented by the formula (1-1) or the structure represented by the formula (1-2). For example, the particle may be obtained by causing a titanium oxide-containing particle and a monomer to coexist in an aqueous medium and adding a polymerization initiator thereto to perform a polymerization reaction, that is, by performing seed polymerization. The monomer is not particularly limited, but for example, the particle may be obtained by performing seed polymerization using glycidyl (meth)acrylate as the monomer and then performing the ring-opening reaction of a glycidyl group. In addition, the monomers may be used alone or as a mixture thereof. The monomer to be mixed is not particularly limited, but when the monomer has the structure represented by the formula (2) or the structure represented by the formula (3), the particle is obtained by mixing monomers from which the structure represented by the formula (2) or the structure represented by the formula (3) is derived and performing seed polymerization by copolymerization of two or more kinds of monomers. The monomer from which the structure represented by the formula (2) or the structure represented by the formula (3) is derived is not particularly limited as long as the monomer has a structure from which the structure represented by the formula (2) or the structure represented by the formula (3) is derived. Examples thereof include vinyltrimethoxysilane, vinyltriethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, and 3-acryloxypropyltrimethoxysilane. Those monomers may also be used alone or in combination thereof.

In addition, the particle for immunoturbidimetry of the present disclosure is not particularly limited as long as the particle includes titanium oxide in the second layer, but the particle may include a third layer containing a second resin inside the second layer containing titanium oxide. The second resin of the present disclosure is not particularly limited, but it is preferred that the second resin have a high refractive index (1.5 or more). Preferred specific structures for that purpose may include a styrene structure, a fluorene structure, a dinaphthothiophene structure, a naphthalene structure, an anthracene structure, and a phenanthrene structure.

The particle including the third layer containing the second resin inside the second layer containing titanium oxide may be obtained, for example, by causing the second resin, a metal oxide precursor, an oxygen-containing organic solvent, and water to coexist to perform a hydrolysis reaction, that is, by a sol-gel method.

A preferred range of the volume average particle diameter of the particles for immunoturbidimetry according to the present disclosure is 150 nm or more and 1,000 nm or less, more preferably 180 nm or more and 800 nm or less. In addition, a preferred range of the particle size distribution of the particle for immunoturbidimetry is a value of the ratio “volume average particle diameter/number average particle diameter” of 1.00 or more and 1.25 or less.

The content of titanium oxide in the particle for immunoturbidimetry according to the present disclosure is preferably 10 mass % or more and 98 mass % or less, more preferably 20 mass % or more and 90 mass % or less. When the content of titanium oxide is 98 mass % or less, the titanium oxide is less liable to be exposed, and non-specific adsorption can be further suppressed. In addition, when the content is 10 mass % or more, the effect of the incorporation of titanium oxide is sufficiently exhibited, and the detection of a trace component in a low-concentration region is excellent.

The particle for immunoturbidimetry according to the present disclosure may be a pre-sensitization particle to which no ligand is imparted, or may be an affinity particle further including a ligand on its surface. The affinity particle has selectively or specifically high affinity to a target substance by virtue of the ligand added to the surface of the particle. In particular, it is preferred that the ligand be added to the surface of the particle by a chemical bond.

The ligand in the present disclosure is a compound that specifically binds to a receptor for a specific target substance. A site where the ligand binds to the target substance is fixed, and has selectively or specifically high affinity. Examples thereof include an antigen and an antibody, an enzyme protein and a substrate therefor, a signal substance, such as a hormone or a neurotransmitter, and a receptor therefor, and a nucleic acid, but the ligand in the present disclosure is not limited thereto. An example of the nucleic acid is deoxyribonucleic acid. The affinity particle in the present disclosure has selectively or specifically high affinity to the target substance. It is preferred that the ligand in the present disclosure be any one of an antibody, an antigen, and a nucleic acid.

In addition, a reagent according to the present disclosure is a reagent in which the particle for immunoturbidimetry according to the present disclosure is dispersed in an aqueous solution.

The test object of the reagent is not particularly limited. An example of the reagent is an in vitro diagnostic drug, and examples thereof include an antigen detection reagent using an antigen as a test object and an antibody detection reagent using an antibody as a test object. The reagent may be a reagent in which a pre-sensitization particle free of a ligand is dispersed in an aqueous solution, assuming that a user adds a desired ligand such as an antibody to the particle. Alternatively, assuming a specific target substance, the reagent may be such a reagent that an affinity particle, in which a ligand has been added, is dispersed in an aqueous solution in advance.

The reagent according to the present disclosure includes the particle for immunoturbidimetry (pre-sensitization particle or affinity particle) according to the present disclosure and a dispersion medium for dispersing the particle for immunoturbidimetry. The reagent according to the present disclosure may include a third substance, such as a solvent or a blocking agent, in addition to the particle for immunoturbidimetry according to the present disclosure to the extent that the object of the present disclosure can be achieved. Two or more kinds of third substances, such as a solvent and a blocking agent, may be incorporated in combination. Examples of the dispersion medium to be used include various buffers, such as a phosphate buffer, a glycine buffer, a Good's buffer, a Tris buffer, and an ammonia buffer, but the dispersion medium in the reagent is not limited thereto.

A test kit according to the present disclosure includes the reagent according to the present disclosure and a container that accommodates the reagent. In addition to the reagent (hereinafter referred to as “reagent 1”) described above, the test kit may include a reaction buffer (hereinafter referred to as “reagent 2”). A sensitizer may be incorporated into each of both the reagent 1 and the reagent 2, or any one of the reagents. In addition, the test kit in the present disclosure may include a positive control, a negative control, a serum diluent, a primary antibody, a secondary antibody, and the like in addition to the reagent 1 and the reagent 2. As a medium for the positive control or the negative control, a solvent may be used in addition to serum and physiological saline each free of a target substance that may be measured.

A method of detecting a target substance according to the present disclosure may be a method of detecting a target substance in a specimen by in vitro diagnosis, the method including mixing the reagent according to the present disclosure and a specimen that may contain the target substance. In addition, the method of detecting a target substance according to the present disclosure may be a method of detecting a target substance in a specimen by in vitro diagnosis, the method including: mixing the reagent according to the present disclosure and a specimen that may contain the target substance to provide a mixed solution; irradiating the mixed solution with light; and detecting at least one of transmitted light and scattered light from the light with which the mixed solution has been irradiated. (Method of measuring Content of Titanium Oxide in Particle for Immunoturbidimetry)

An example of a method of measuring the content of titanium oxide in the particle for immunoturbidimetry in the present disclosure is described. The content of titanium oxide in the particle is measured with a thermogravimetric analyzer. For example, STA200 (manufactured by Hitachi High-Tech Science Corporation) is used. 5 Milligrams to 10 mg of a cured product is weighed in an aluminum pan and measured in a nitrogen atmosphere. The temperature condition is to hold the temperature at 30° C. for 30 minutes, then increase the temperature from 30° C. to 500° C. at 10° C./min, and subsequently hold the temperature at 500° C. for 10 minutes. The content of titanium oxide in the particle may be measured from the mass loss of a resin component at 300° C. (Methods of measuring Volume Average Particle Diameter and Particle Size Distribution of Particles for Immunoturbidimetry in Aqueous Dispersion)

An example of a method of measuring the volume average particle diameter (Dv) of the particles for immunoturbidimetry in the present disclosure is described. The Dv of particles present in an aqueous dispersion is measured by a dynamic light scattering method. For example, a Zetasizer (Zetasizer ultra: manufactured by Malvern Panalytical Ltd.) is used to measure the Dv at 25° C.

In addition, the particle size distribution of the particles for immunoturbidimetry in the present disclosure is calculated by measuring a number average particle diameter (Dn) by the above-mentioned dynamic light scattering method to determine the ratio of Dv to Dn (Dv/Dn).

### EXAMPLES

The present disclosure is described in detail below by way of Examples, but the present disclosure is not limited to these Examples.

### Particle Preparation Example

### (Step of Forming Third Layer Containing Resin)

12.68 Grams of styrene (St: Kishida Chemical Co., Ltd.), 0.23 g of divinylbenzene (DVB: Kishida Chemical Co., Ltd.), and 1,512.02 g of ion-exchanged water were weighed into a 2 L four-necked separable flask to prepare a mixed solution. Oxygen was removed from the inside of the four-necked separable flask by holding the mixed solution at 70° C. under stirring at 140 rpm and performing a nitrogen flow at a flow rate of 200 ml/min. Next, a dissolved solution of 0.55 g of V-50 (Fujifilm Wako Pure Chemical Corporation) dissolved in 20 g of ion-exchanged water, which had been separately prepared, was added to the mixed solution to start soap-free polymerization. A dispersion of a third layer-forming resin particle 1 containing a copolymer of styrene and divinylbenzene was obtained by a reaction for 23 hours from the start of the polymerization. Part of the dispersion was collected and evaluated by using dynamic light scattering (Zetasizer ultra: Malvern Panalytical Ltd.), and as a result, the volume average particle diameter was 190 nm.

### (Step of Forming Second Layer Containing Titanium Oxide)

The third layer-forming resin particle 1 was adjusted with ion-exchanged water to have a solid content concentration of 0.6%. 20 Grams of the dispersion was mixed into 404.50 g of ethanol (Kishida Chemical Co., Ltd.) containing 0.2% of polyvinylpyrrolidone K-30 (PVP K-30: Kishida Chemical Co., Ltd.), and the mixture was held at 70° C. while being stirred at 140 rpm. Next, a solution of 5.0 ml of titanium(IV) n-butoxide, monomer (TBOT: Kishida Chemical Co., Ltd.) mixed into 197.50 g of ethanol, which had been separately prepared, was added to the mixed solution to start a sol-gel reaction. The reaction was performed for 24 hours from the start of the sol-gel reaction, and the particle dispersed in the mixed solution was separated with a centrifuge and re-dispersed in ethanol. Further, the operation of separating the particle from the dispersion with a centrifuge and re-dispersing the particle in ion-exchanged water was repeated twice for purification to provide a second layer-forming particle 1. The second layer-forming particle 1 was stored in a state of an aqueous dispersion finally adjusted to 5.0 mass %. Part of the dispersion was collected and the dynamic light scattering of the second layer-forming particle 1 was evaluated, and as a result, the volume average particle diameter was 200 nm. In addition, when the metal oxide content was evaluated by using differential thermal-thermogravimetric simultaneous analysis (NEXTA STA200RV: Hitachi High-Tech Corporation), the content was 45% of the particle weight.

### (Step of Forming First Layer Containing Resin)

Adjustment was performed with ion-exchanged water to provide 149.55 g of the second layer-forming particle 1 dispersion having a solid content concentration of 0.2%. 0.135 Gram of glycidyl methacrylate (GMA: Kishida Chemical Co., Ltd.) and 0.015 g of 3-methacryloxypropyltrimethoxysilane (MPS: Shin-Etsu Chemical Co., Ltd.) were added and held at 70° C. while being stirred at 100 rpm. Oxygen was removed from the inside of the four-necked separable flask by performing a nitrogen flow at a flow rate of 200 ml/min. Then, a dissolved solution of 0.03 g of V-50 dissolved in 0.3 g of ion-exchanged water, which had been separately prepared, was added to the mixed solution to start shell formation. A dispersion containing a first layer-forming particle 1 was obtained by continuing stirring for 18 hours after the start of the reaction.

### (Step of Imparting Reactive Functional Group)

An aqueous solution in which mercaptosuccinic acid (MSA: Wako Pure Chemical Industries, Ltd.) was dissolved (the total number of moles of MSA was equal to the number of moles of the glycidyl methacrylate), which had been prepared in advance, was added to the dispersion containing the first layer-forming particle 1. Triethylamine (Kishida Chemical Co., Ltd.) was added thereto to adjust the pH to 10. Next, the mixture was heated to 70° C. while being stirred at 800 rpm and further held in this state for 18 hours to provide a dispersion of the particle. The particle was separated from the dispersion with a centrifuge, and the operation of re-dispersing the particle in ion-exchanged water was repeated eight times to purify the particle to provide a particle 1. The particle 1 was finally stored in the state of an aqueous dispersion adjusted to 5.0 mass %.

### (Preparation of Particle 2)

In the step of forming a first layer containing a resin, 0.15 g of glycidyl methacrylate was added instead of the addition of 0.135 g of glycidyl methacrylate and 0.015 g of 3-methacryloxypropyltrimethoxysilane. A dispersion of a particle 2 was obtained by the same operation as that for the particle 1 except for the foregoing.

### (Preparation of Particle 3)

0.6 Gram of a titanium oxide particle TTO-55 (Ishihara Sangyo Kaisha, Ltd.), 10.1 g of 28% ammonia water (Kanto Chemical Co., Inc.), 45 g of ethanol (Kishida Chemical Co., Ltd.), 44 g of pure water, and 0.3 g of 3-methacryloxypropyltrimethoxysilane (MPS: Shin-Etsu Chemical Co., Ltd.) were mixed. TK Homomixer (PRIMIX Corporation) was used to disperse the particle for 1 hour at 12,000 rpm to provide a titanium oxide fine particle dispersion. The titanium oxide fine particle and the supernatant were separated from the titanium oxide fine particle dispersion with a centrifuge, and ion-exchanged water having the same mass as that of the supernatant was further added for re-dispersion to provide a purified titanium oxide fine particle dispersion.

### (Step of Forming First Layer Containing Resin)

The purified titanium oxide fine particle dispersion adjusted in concentration by adding 80 g of pure water was deoxygenated by a nitrogen flow, and 1.74 g of styrene (St: Kishida Chemical Co., Ltd.) and 0.174 g of divinylbenzene (DVB: Kishida Chemical Co., Ltd.) were added thereto, and the temperature was increased to 70° C. A dissolved solution of 0.04 g of ammonium persulfate (Kishida Chemical Co., Ltd.) dissolved in 1 g of ion-exchanged water was added to the mixed solution to start formation of a styrene layer. After stirring had been continued for 18 hours from the start of the reaction, 0.3 g of glycidyl methacrylate and a dissolved solution of 0.01 g of ammonium persulfate (Kishida Chemical Co., Ltd.) dissolved in 1 g of ion-exchanged water were further added thereto. The reaction was performed for an additional 12 hours. Thus, a dispersion containing a first layer-forming particle 3 was obtained.

### (Impartment of Reactive Functional Group)

A dispersion of a particle 3 was obtained by the same method as that for the particle 1 except that the first layer-forming particle 3 was used instead of the first layer-forming particle 1.

The obtained particles 1 to 3 each have the structure represented by the formula (1-A-9).

### Comparative Particle Preparation Example

### (Preparation of Comparative Particle)

Titanium tetraethoxide was added to an acetonitrile/ethanol solution to prepare a 0.1 M titanium tetraethoxide solution. Ethanol and 0.1 M ammonia water were mixed into the solution, and the mixture was stirred at room temperature for 60 minutes so that hydrolysis was sufficiently performed. After the hydrolysis, the mixture was stirred at 80° C. for 3 hours or more, heated to reflux, and then centrifugally washed and adjusted in concentration to about 20% of a solid component to provide a titanium oxide particle dispersion. Next, 5 ml of water was added to 1 g of a copolymer of polyoxyethylene-monoallyl-monomethyl ether and maleic anhydride (NOF Corporation). The solution obtained after hydrolysis and 1-ethyl-3-(3-dimethylaminopropyl)carbodiimide hydrochloride were mixed by using ultrapure water so that the concentrations were 50 mg/ml and 50 mM, respectively. 4-Aminosalicylic acid (Wako Pure Chemical Industries, Ltd.) was mixed into the prepared solution to a concentration of 0.1 M, and the mixture was shaken and stirred at room temperature for 24 hours to be subjected to a reaction. After the reaction, the obtained solution was transferred to a Spectra/Por CE dialysis tube (molecular weight cut-off=3,500, Spectrum Laboratories, Inc.) and dialyzed at room temperature for 24 hours. After the dialysis, dimethylformamide (DMF: Wako Pure Chemical Industries, Ltd.) was added to the powder obtained by freeze-drying to a concentration of 25 mg/ml and mixed to provide a 4-aminosalicylic acid-bonded PEG solution. Next, DMF was used to adjust the 4-aminosalicylic acid-bonded PEG solution to a final concentration of 0.6 mg/ml and the titanium oxide particle dispersion to a final concentration of 0.5% of the solid component to provide a 20 ml reaction solution.

This reaction solution was subjected to a heating reaction for 16 hours at 130° C. After the completion of the reaction, the reaction container was cooled to 50° C. or less, DMF was removed with an evaporator, and then distilled water was added to obtain a comparative particle in which a dispersant was bonded to the surface of the particle.

### (Preparation of Affinity Particle)

300 Microliters (3 mg in terms of a particle solid content) of the dispersion of the particle 1 diluted with ion-exchanged water to a solid content concentration of 1.0 mass % was aliquoted into a 1.5 mL microtube. 90 Microliters of a 5.0% aqueous solution of 1-(3-dimethylaminopropyl)-3-ethylcarbodiimide hydrochloride (Tokyo Chemical Industry Co., Ltd.) and 90 μL of a 5.0% aqueous solution of N-hydroxysulfosuccinimide sodium salt (Tokyo Chemical Industry Co., Ltd.) were added thereto. The mixture was stirred at room temperature for 30 minutes to provide a dispersion of a particle having an activated carboxy group (activated particle dispersion).

After centrifugal washing, 270 μL of phosphate-buffered saline (hereinafter referred to as “PBS”) having a pH of 5.5 was added, and the particle having an activated carboxy group was dispersed with an ultrasonic wave.

24 Microliters (0.12 mg in terms of an antibody amount) of a 5.0 mg/mL dispersion of a mouse monoclonal anti-ferritin antibody (isoelectric point: 7.1) was added thereto, and the mixture was stirred at room temperature for 3 hours to provide an antibody-sensitized affinity particle 1.

Affinity particles 2 and 3 and a comparative affinity particle were prepared by the same experimental operation as that in the preparation of the affinity particle 1 except that the kind of the particle was changed to the particle 2, the particle 3, and the comparative particle, respectively, in the preparation of the affinity particle 1.

### (Preparation of First Reagent)

A first reagent was obtained by dissolving 50 mM HEPES, 0.05 mass % Triton X-100, and 1.0 mass % sodium chloride (Kishida Chemical Co., Ltd.) in ion-exchanged water.

### (Preparation of Second Reagent)

After the centrifugal washing of the affinity particle 1, the resultant was re-dispersed in 500 μL of a buffer (HEPES buffer) in which 10 mM HEPES, 0.01 mass % polyoxyethylene nonylphenyl ether (Triton X-100, Kishida Chemical Co., Ltd.), and 10 mass % sucrose (viscosity modifier) was dissolved in ion-exchanged water. After that, the contents were mixed and diluted with the HEPES buffer so that the concentration of the affinity particle 1 became 0.1 mass % to provide a second reagent 1.

A second reagent 2, a second reagent 3, and a comparative second reagent were prepared by the same experimental operation as that in the preparation of the second reagent 1 except that the kind of the particle was changed to the affinity particle 2, the affinity particle 3, and the comparative affinity particle, respectively, in the preparation of the second reagent 1.

### (Measurement of Absorbance Change Amount)

A mixed solution was prepared by mixing 15 μL of each of specimens adjusted to have ferritin concentrations of 0.0 ng/mL (physiological saline) and 100 ng/mL with 60 μL of the first reagent 1, and was kept under the condition of 37° C. for 290 seconds. Next, 30 μL of each of the second reagents 1 to 3 and the comparative second reagent was mixed into the mixed solution, and the mixture was stirred, and the absorbance 42 seconds later was measured. Further, the mixed solution was left to stand still at 37° C. for 253 seconds, and the absorbance was measured again, and the difference from the absorbance 42 seconds after the stirring was defined as an absorbance change amount ΔABS. The absorbance was measured at a measurement wavelength of 572 nm with a spectrophotometer BIOSPECTROMETER manufactured by Eppendorf Co. As a result, all the second reagents showed a difference between the absorbance change amount ΔABS at a ferritin concentration of 0.0 ng/mL (physiological saline) and the absorbance change amount ΔABS at 100 ng/mL, and showed satisfactory results.

### (Calculation of Water Dispersion Stability Index)

A mixed solution was prepared by mixing 15 μL of a chylomicron solution containing triolein, lecithin, a free fatty acid, bovine albumin, and a Tris buffer with 60 L of the first reagent 1, and was kept under the condition of 37° C. for 290 seconds. Next, L of each of the second reagents 1 to 3 and the comparative second reagent was mixed into the mixed solution, and the mixture was stirred, and the absorbance 42 seconds later was measured. Further, those mixed solutions were left to stand still at 37° C. for 253 seconds, and the absorbance was measured again, and the difference from the absorbance 42 seconds after the stirring was defined as the absorbance change amount ΔABS. The absorbance was measured at a measurement wavelength of 572 nm with a spectrophotometer BIOSPECTROMETER manufactured by Eppendorf Co. After each second reagent had been stored at 4° C. for 30 days, the same evaluation was performed, and a water dispersion stability index was calculated by using the initial absorbance change amount ΔABS (initial) and the absorbance change amount ΔABS (30 days later) using the following equation. The physical properties and evaluation of the water dispersion stability index of the affinity particle used in each of Examples and Comparative Example are shown in Table 1.

Water dispersion stability index=|ΔABS(30 days later)−ΔABS(initial)|/ΔABS (initial)

The evaluation was performed as described below depending on the value of the water dispersion stability index.
A: The water dispersion stability index was less than 0.05.
B: The water dispersion stability index was 0.05 or more and less than 0.20.
C: The water dispersion stability index was 0.20 or more.

TABLE 1

| | | | | Comparative |
|---|---|---|---|---|
| | Example 1 | Example 2 | Example 3 | Example 1 |
| Affinity particle | Affinity | Affinity | Affinity | Comparative |
| | particle 1 | particle 2 | particle 3 | affinity particle |
| Content of | 32% | 32% | 10% | 40% |
| titanium oxide | | | | |
| Volume average | 200 nm | 200 nm | 200 nm | 150 nm |
| particle diameter | | | | |
| Water dispersion | A | B | B | C |
| stability | | | | |

As described above, the particles 1 to 3 for immunoturbidimetry of the present disclosure are each excellent in water dispersion stability. In contrast, Comparative Example 1 had low water dispersion stability.

According to the present disclosure, the particle for immunoturbidimetry including titanium oxide, which detects a trace component in a low-concentration region and suppresses a change in water dispersion stability over time, can be provided.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-208571, filed Nov. 29, 2024, which is hereby incorporated by reference herein in its entirety.

What is claimed is:

1. A particle for immunoturbidimetry comprising a first resin and titanium oxide,
wherein the particle for immunoturbidimetry includes a first layer containing the first resin and a second layer containing the titanium oxide,
wherein the second layer is arranged inside the first layer, and
wherein the first resin has at least one of a structure represented by the following formula (1-1) and a structure represented by the following formula (1-2): 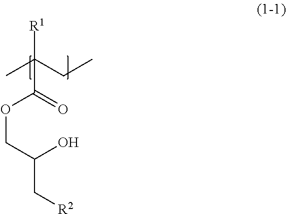
in the formula (1-1), R¹ represents a hydrogen atom or a methyl group, and R² represents a structure having a hydroxy group or a structure having a carboxy group; and 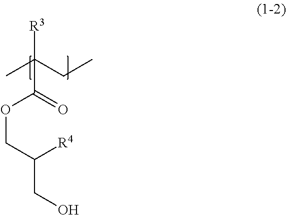
in the formula (1-2), R³ represents a hydrogen atom or a methyl group, and R⁴ represents a structure having a hydroxy group or a structure having a carboxy group.

2. The particle for immunoturbidimetry according to claim 1, wherein the first resin further has at least one of a structure represented by the following formula (2) and a structure represented by the following formula (3): 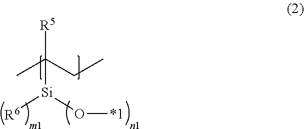
in the formula (2), R⁵ represents a hydrogen atom or a methyl group, n1 represents an integer of 1 or more and 3 or less, m1 represents an integer of 0 or more and 2 or less, n1+m1 is 3, *1s each independently represent a bond to a titanium atom or a silicon atom, or represent a hydrogen atom, a methyl group, or an ethyl group, and R⁶s each independently represent a methyl group or an ethyl group; and 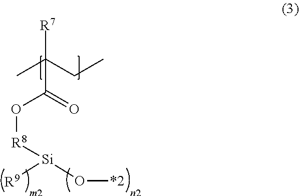
in the formula (3), R⁷ represents a hydrogen atom or a methyl group, R⁸ represents a single bond, a phenylene group, or an alkylene group having 3 or less carbon atoms, n2 represents an integer of 1 or more and 3 or less, m2 represents an integer of 0 or more and 2 or less, n2+m2 is 3, *2s each independently represent a bond to a titanium atom or a silicon atom, or represent a hydrogen atom, a methyl group, or an ethyl group, and R⁹s each independently represent a methyl group or an ethyl group.

3. The particle for immunoturbidimetry according to claim 1, wherein a content of the titanium oxide in the particle for immunoturbidimetry is 10 mass % or more and 98 mass % or less.

4. The particle for immunoturbidimetry according to claim 1, wherein the particle for immunoturbidimetry has a volume average particle diameter of 150 nm or more and 1,000 nm or less.

5. The particle for immunoturbidimetry according to claim 1, wherein the particle for immunoturbidimetry includes a third layer containing a second resin inside the second layer containing the titanium oxide.

6. The particle for immunoturbidimetry according to claim 1, further comprising a ligand on a surface thereof.

7. A reagent comprising the particle for immunoturbidimetry of claim 1 and an aqueous solution,
wherein the particle for immunoturbidimetry is dispersed in the aqueous solution.

8. A test kit comprising the reagent of claim 7 and a container configured to accommodate the reagent.

9. A method of detecting a target substance in a specimen by in vitro diagnosis, the method comprising mixing the reagent of claim 7 and a specimen that may contain the target substance.

10. A method of detecting a target substance in a specimen by in vitro diagnosis, the method comprising:
mixing the reagent of claim 7 with a specimen that may contain the target substance to provide a mixed solution;
irradiating the mixed solution with light; and
detecting at least one of transmitted light and scattered light from the light with which the mixed solution has been irradiated.

11. A particle for immunoturbidimetry comprising a first resin and titanium oxide,
wherein the particle for immunoturbidimetry includes a first layer containing the first resin and a second layer containing the titanium oxide,
wherein the second layer is arranged inside the first layer, and
wherein the first resin is a vinyl-based polymer that has one of a carboxy group and a hydroxy group in a side chain thereof and forms a hydrogen bond with the titanium oxide in the second layer.
